(19) United States
(12) Patent Application Publication
(10) Pub. No.: US 2026/0083680 A1
(43) Pub. Date: Mar. 26, 2026

(54) MESOPOROUS NANO-MAGNESIUM OXIDE FOR DRUG LOADING AND PREPARATION METHOD THEREOF

(71) Applicant: SHANGHAI INSTITUTE OF TECHNOLOGY, Shanghai (CN)

(72) Inventor: Chun XU, Shanghai (CN)

(73) Assignee: SHANGHAI INSTITUTE OF TECHNOLOGY, Shanghai (CN)

(21) Appl. No.: 19/302,131

(22) Filed: Aug. 18, 2025

(30) Foreign Application Priority Data
Sep. 24, 2024 (CN) 202411331395.3

(51) Int. Cl. A61K 9/51

(52) U.S. Cl. CPC A61K 9/5115; A61K 9/5192

(57) ABSTRACT

A mesoporous nano-magnesium oxide for drug loading and a preparation method thereof are provided. The mesoporous nano-magnesium oxide particle has a particle size of 50 nm to 150 nm, and includes abundant mesoporous structures with a pore size of 2 nm to 20 nm. A surface of the particle has a positive potential in absolute ethanol, with a Zeta potential distribution of 10 mV to 100 mV. The preparation method includes: preparing a mixture of magnesium oxalate and CTAB as a precursor; adding the precursor to a quartz crucible, and placing the quartz crucible in a muffle furnace; calcining at 200℃ for 1 h to make the CTAB in the mixture completely decomposed to produce pure magnesium oxalate; and heating to 530°C, and calcining for 1 h to 6 h to make the magnesium oxalate fully decomposed to produce the nano-magnesium oxide microparticle.

### CROSS REFERENCE TO THE RELATED APPLICATIONS

This application is based upon and claims priority to Chinese Patent Application No. 202411331395.3, filed on September 24, 2024, the entire contents of which are incorporated herein by reference.

### TECHNICAL FIELD

The present disclosure relates to a material belonging to the biology field, and specifically relates to a magnesium oxide nanomaterial that can be used as a drug carrier. The magnesium oxide nanomaterial is suitable for the production of targeted carriers for anti-tumor drugs or drug carriers for other uses in the human body, and possesses an auxiliary pharmacological effect.

### BACKGROUND

A nanodrug carrier can deliver one or more drugs simultaneously. Nanodrug carriers can not only improve the solubility and stability of drugs but also regulate the release rate of drugs. Moreover, nanodrug carriers enable the active or passive targeting of drugs for diseased tissues and cells, particularly tumors, thereby enhancing the utilization efficiency of drugs while reducing the toxic side effects of drugs for normal cells or tissues.

Currently, there are many types of nanodrug carriers. Generally, inorganic nanoparticles have a stable structure, such as the traditional nanoparticles of gold, silver, copper, and iron and oxides thereof. However, nanocarriers produced from these elements have large toxic side effects. Magnesium oxide is easily metabolized and has minimal toxic side effects. As a result, magnesium oxide has a promising potential for future applications in the field of drug carriers. However, currently, nano-magnesium oxide, as a drug carrier, has disadvantages such as large particle size and size non-uniformity. The patent CN108619518A provides a method for preparing a micro-nano magnesium oxide drug carrier. The method is as follows: MgCO₃ is mixed with oxalic acid to produce a magnesium oxalate hydrate intermediate, MgC₂O₄·nH₂O, and the magnesium oxalate hydrate intermediate is calcined to produce magnesium oxide with micro-nano structures. The magnesium oxide has an average particle size of 2.3 μm and the magnesium oxide with micro-nano structures includes merely about 5% or less of nano-scale structures. Although the patent CN114477246A provides a preparation method of spherical nano-magnesium oxide with a particle size of about 40 nm, the spherical nano-magnesium oxide is mainly used as a thermally conductive filler to improve the thermal conductivity of a corresponding composite. The preparation method is as follows: Magnesium acetate tetrahydrate and cetyltrimethylammonium bromide (CTAB) are dissolved in ethanol to produce a magnesium acetate-containing ethanol solution. Oxalic acid is dissolved in deionized water to produce an oxalic acid solution. The magnesium acetate-containing ethanol solution and the oxalic acid solution are subjected to continuous ultrasonic stirring. Centrifugation is then conducted multiple times with deionized water and ethanol. Then drying is conducted at 60°C to 90°C for 8 h to 12 h. Calcination is conducted at 550°C to 650°C for 2 h to 4 h. In this patent, the surface potential and surface morphology are not considered. The patent CN108996530B discloses a preparation method of nano-magnesium oxide. The nano-magnesium oxide is prepared with magnesium nitrate and sodium carbonate as raw materials. The nano-magnesium oxide has a particle size of 2 nm to 22 nm. The prepared nano-magnesium oxide is mainly used in the field of decontamination and primarily provides a degradation effect for paraoxon. The above patents evidently overlook factors such as physicochemical properties, sizes, geometric shapes, surface potentials, and surface ligand shapes of nanodrug carriers, which affect the use effects of nanodrug carriers. The purpose of the present disclosure is to design a nanodrug carrier with specific physicochemical properties that can effectively improve the delivery efficiency of nanodrug loading systems, thereby improving the efficacy and reducing the toxic side effects of drugs.

### SUMMARY

In order to overcome the problems such as instable physicochemical properties and large toxic side effects of the existing nanodrug carriers, the present disclosure provides mesoporous nano-magnesium oxide for drug loading and a preparation method thereof. A main component of the mesoporous nano-magnesium oxide carrier is magnesium oxide. The mesoporous nano-magnesium oxide has a specific size and a large number of micropores and mesopores on a surface, which facilitates the loading of anti-tumor drugs. Moreover, the mesoporous nano-magnesium oxide can respond sensitively to a pH value, can be quickly dissolved in an acidic environment within a tumor, and can also provide auxiliary effects such as anti-inflammatory and analgesic effects.

To solve the technical problem, the present disclosure adopts the following technical solutions: Mesoporous nano-magnesium oxide for drug loading is provided. The particle of the mesoporous nano-magnesium oxide for drug loading has a particle size of 50 nm to 150 nm, and includes abundant mesoporous structures on a surface with a pore size of 2 nm to 20 nm.

Further, a surface of the particle of the mesoporous nano-magnesium oxide for drug loading has a positive potential in absolute ethanol, with a Zeta potential distribution of 10 mV to 100 mV.

A preparation method of the mesoporous nano-magnesium oxide for drug loading is provided, including the following steps:

(1) preparation of a magnesium oxalate precursor:

with magnesium chloride hexahydrate as a magnesium source, oxalic acid dihydrate as a precipitating agent, a mixture of water and n-butanol as a solvent, CTAB as a dispersing agent, and acetic acid added to increase a hydrogen ion concentration, conducting a co-precipitation reaction to synthesize the magnesium oxalate precursor, where the precursor is a mixture of magnesium oxalate and the CTAB; and

(2) preparation of a nano-magnesium oxide microparticle

adding the precursor mixture to a quartz crucible, and placing the quartz crucible in a muffle furnace; calcining at 200℃ for 1 h to make the CTAB in the mixture completely decomposed to produce pure magnesium oxalate; and heating to 530°C, and calcining for 1 h to 6 h to make the magnesium oxalate fully decomposed to produce the nano-magnesium oxide microparticle.

Further, a concentration ratio of the magnesium chloride hexahydrate to the oxalic acid dihydrate is (1-3):(3-1); and a mixed solvent of deionized water and the n-butanol in a volume ratio of (1-3):(1-10) is added.

Further, during the co-precipitation reaction synthesis, 0.25 wt% to 1.25 wt% of the CTAB and the acetic acid with a concentration of 36% to 50% are added to improve dispersibility of the magnesium oxalate.

Further, a specific process for preparing the magnesium oxalate precursor is as follows: placing a beaker with an appropriate amount of the solvent in a water bath; when a temperature reaches a predetermined reaction temperature, adding a specified concentration ratio of the magnesium chloride hexahydrate and an appropriate amount of the CTAB, thoroughly stirring, and adding specified amounts of the oxalic acid dihydrate and the acetic acid; using an alcohol and deionized water as detergents; conducting decantation multiple times, and retaining a lower precipitate; and oven-drying to produce the magnesium oxalate precursor.

Further, the reaction temperature is 40°C to 80°C, and a reaction time is 20 min to 60 min; the stirring is continuously conducted for 20 min under heating with a magnetic stirrer at a rotational speed of 100 r/min to 1,000 r/min; and after the decantation is conducted multiple times, the drying is conducted at 90°C to 100°C for 1 h.

Further, with the decomposition of the magnesium oxalate, a gas is released from a surface of the nano-magnesium oxide microparticle to form a large number of mesopores and micropores, with an average pore size of 14 nm and a pore size distribution of 2 nm to 20 nm.

Compared with the prior art, the present disclosure has the following advantages:

(1) Compared with the existing drug carrier materials, magnesium oxide itself causes minimal toxic side effects to the human. Magnesium oxide has long been used as a gastric acid-neutralizing agent widely in clinical practice. Some studies on the toxicity of magnesium oxide show that, within a considerable dose range, magnesium oxide exhibits high biosafety. Nano-magnesium oxide enables higher drug permeability in vivo than micro-magnesium oxide. Nano-magnesium oxide can be metabolized fast, and resulting metabolites are relatively safe.

(2) In contrast to the magnesium oxide currently sold on the market, the magnesium oxide synthesized by the method of the present disclosure has a concentrated particle size distribution, a regular shape, and micropores and mesopores densely distributed on a surface, primarily mesopores. In absolute ethanol, a surface of the magnesium oxide of the present disclosure carries positive charges, which are conducive to the drug loading and allow the efficient drug delivery.

(3) Compared with the existing nano-magnesium oxide produced with the same type of technology, the present disclosure adjusts the process parameters of the experiment and optimizes the dispersion process to produce magnesium oxide with a particle size (50 nm to 150 nm) suitable as a drug carrier. The present disclosure also weakens the aggregation of nano-magnesium oxide microparticles, and makes the product suitable for the delivery of anti-tumor drugs.

(4) The magnesium source adopted in the present disclosure is magnesium chloride hexahydrate, which has large reserves in seawater and is easily available. Other compounds adopted in the present disclosure are also common and have relatively-mature production processes. In addition, the present disclosure involves a simple preparation flow, a short production time, and an easily-controlled preparation process. Therefore, the present disclosure has a high production efficiency and a low cost.

### BRIEF DESCRIPTION OF THE DRAWINGS

FIG. 1 shows a Brunauer-Emmett-Teller (BET) adsorption curve of a mesoporous nano-magnesium oxide carrier for a targeted drug;

FIG. 2 shows morphologies of mesoporous nano-magnesium oxide under a scanning electron microscope (SEM);

FIG. 3 shows a microscopic morphology of nano-magnesium oxide prepared in Example 1;

FIG. 4 shows a particle size distribution of the nano-magnesium oxide prepared in Example 1;

FIG. 5 shows nitrogen adsorption-desorption curves of the nano-magnesium oxide prepared in Example 1; and

FIG. 6 shows a pore size distribution of the nano-magnesium oxide prepared in Example 1.

### DETAILED DESCRIPTION OF THE EMBODIMENTS

The present disclosure is further explained below in conjunction with the accompanying drawings and embodiments, but the present disclosure is not limited accordingly to the scope of the embodiments.

The present disclosure provides a mesoporous nano-magnesium oxide for drug loading. The particle of the mesoporous nano-magnesium oxide has a particle size of 50 nm to 150 nm, includes abundant mesoporous structures on a surface with a pore size of 2 nm to 20 nm. A surface of the particle of the mesoporous nano-magnesium oxide has a positive potential in absolute ethanol, with a Zeta potential distribution of 10 mV to 100 mV.

The present disclosure also provides a preparation method of the mesoporous nano-magnesium oxide for drug loading, including:

(1) Preparation of a magnesium oxalate precursor:

With magnesium chloride hexahydrate as a magnesium source, oxalic acid dihydrate as a precipitating agent, and a mixture of water and n-butanol as a solvent, a co-precipitation reaction is conducted to synthesize a precursor magnesium oxalate microparticle. Experimental parameters are adjusted to produce a magnesium oxalate nanoparticle with a specific particle size (100 nm to 200 nm). CTAB is added to produce colloidal bundles. The mixture of n-butanol and water is adopted as a solvent to reduce a dielectric constant of the solvent. Acetic acid is added to adjust a pH to change the surface charges of nanoparticles, thereby improving the dispersibility of the produced nanoparticles.

A molar ratio of magnesium chloride to oxalic acid is (1-3):(1-3). The co-precipitation reaction is conducted at 40°C to 80°C for 20 min to 60 min. The stirring is conducted under heating by a magnetic water-bath thermostatic stirring pot to ensure the uniform particle size of the produced microparticles. A mixed solution of deionized water and n-butanol in a volume ratio of (1-3):(1-10) is adopted as the solvent. A specific process was as follows: A beaker with an appropriate amount of the solvent is placed in a water bath. When a temperature reaches a predetermined reaction temperature, a specified concentration ratio of the magnesium chloride hexahydrate and an appropriate amount of the CTAB are added, and stirring is conducted thoroughly for 20 min under heating at a rotational speed of 100 r/min to 1,000 r/min. Then specified amounts of the oxalic acid dihydrate and the acetic acid are added, and the stirring is conducted thoroughly for 20 min under heating at a rotational speed of 100 r/min to 1,000 r/min. Using an alcohol and deionized water as detergents. Decantation is conducted multiple times, and a lower precipitate is retained and oven-dried at 90°C to 100°C for 1 h to produce the magnesium oxalate precursor.

(2) Preparation of a nano-magnesium oxide microparticle

The precursor is a mixture of magnesium oxalate and the CTAB. The precursor mixture is added to a quartz crucible, placed in a muffle furnace, and calcined at 200℃ for 1 h to make the CTAB in the mixture completely decomposed to produce pure magnesium oxalate. The magnesium oxalate is heated to 530°C, and calcined for 1 h to 6 h to make the magnesium oxalate fully decomposed to produce the nano-magnesium oxide. With the decomposition of magnesium oxalate, a gas is released from a surface of the microparticle to form a large number of pores. According to test results, these pores are mainly mesopores and micropores, with an average pore size of 14 nm.

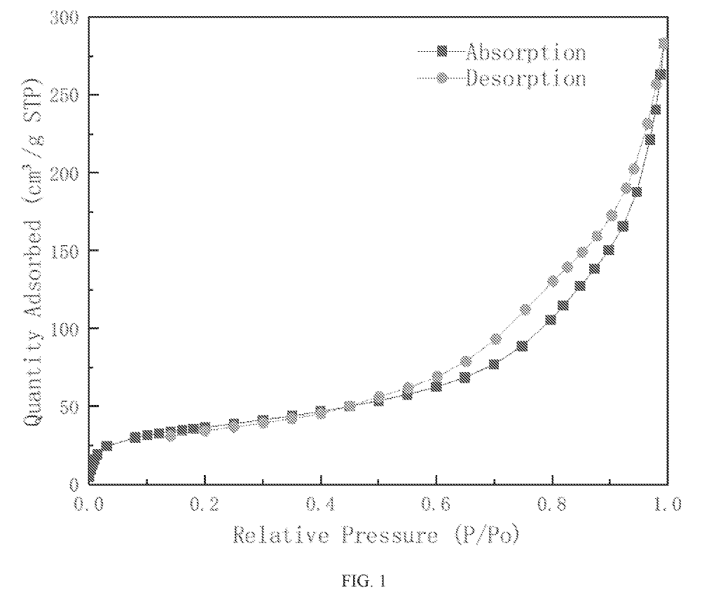

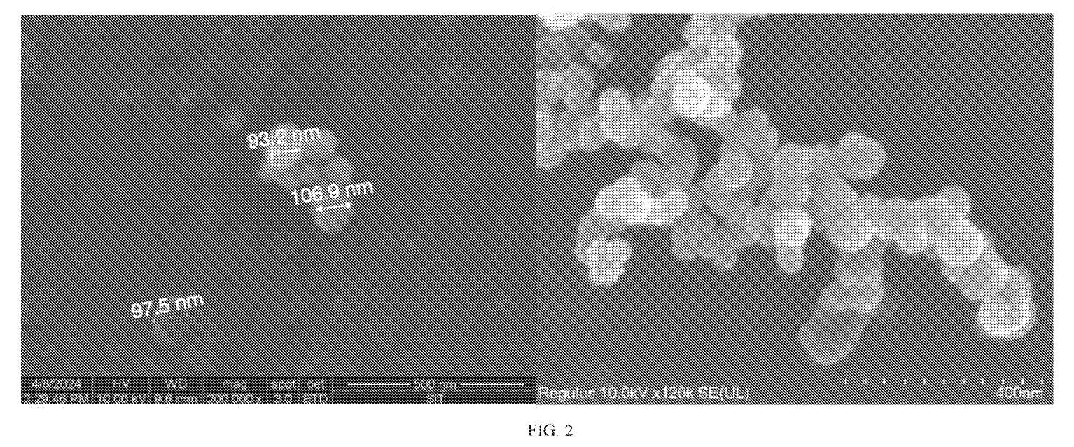

The particle is beneficial for the loading of an anti-tumor drug, exhibits excellent stability in the environment in the human body, and can be quickly dissolved in a slightly-acidic environment with a pH of 5.0. A BET adsorption curve and scanned images of the prepared particle are shown in FIGS. 1 and 2, respectively.

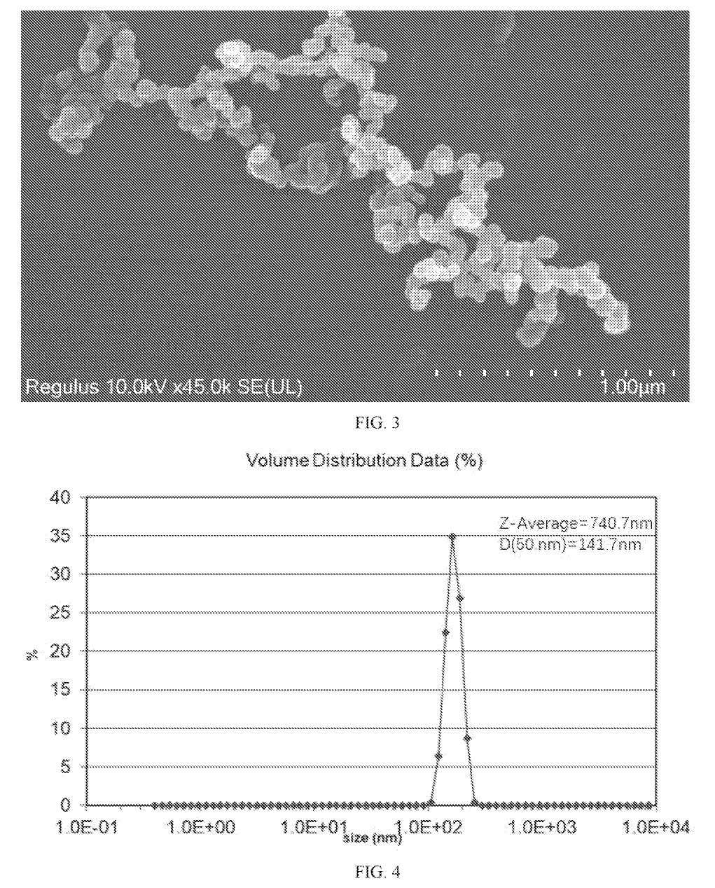

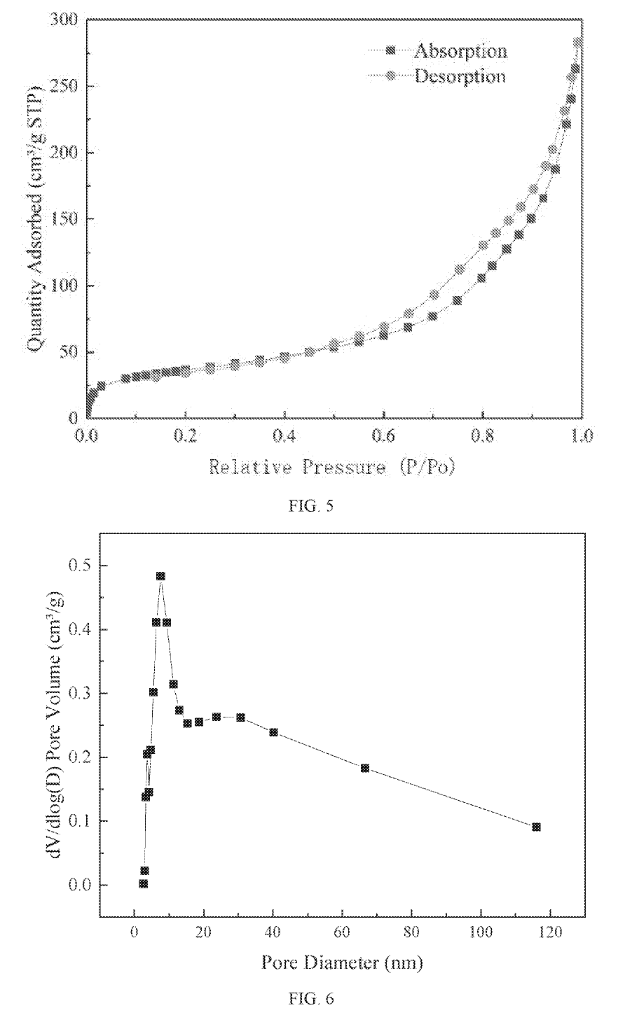

In the following example, magnesium chloride hexahydrate with a purity of 99% or more and oxalic acid with a purity of 99.8% or more are adopted as the main raw materials, and other auxiliary materials also have a purity of 99% or more. A microscopic morphology of the prepared magnesium oxide microparticle is observed by SEM. A particle size distribution and a surface charge distribution of the prepared magnesium oxide microparticle are tested by a laser particle size analyzer. A pore size distribution and a pore volume of the prepared magnesium oxide microparticle are tested through a nitrogen adsorption-desorption test. Characterization results are shown in FIG. 3 to FIG. 6.

### Example

A process of preparing magnesium oxalate through co-precipitation and then calcining the precursor magnesium oxalate for decomposition to produce mesoporous nano-magnesium oxide included the following steps:

1) A beaker with 200 mL of a mixture of deionized water and n-butanol in a volume ratio of 1:2 was placed in a thermostatic magnetic stirring pot. When a temperature reached 60°C, 20.33 g of magnesium chloride hexahydrate and 1.0 g of CTAB were added, and stirring was fully conducted for 20 min under heating at a rotational speed of 800 r/min. Then 12.61 g of oxalic acid dihydrate and acetic acid with a concentration of 36% were added, and stirring was continuously conducted for 20 min under heating at a rotational speed of 800 r/min. Washing was conducted with absolute ethanol and deionized water. Decantation was conducted multiple times, and a lower precipitate was retained and oven-dried at 90°C to 100°C for 1 h to produce a magnesium oxalate precursor.

2) The precursor mixture was added to a quartz crucible, placed in a muffle furnace, and calcined at 200℃ for 1 h to make the CTAB in the mixture completely decomposed to produce pure magnesium oxalate. The magnesium oxalate was heated to 530°C, and calcined for 2 h to make the magnesium oxalate fully decomposed to produce the nano-magnesium oxide.

The produced magnesium oxide was subjected to various characterizations, including SEM, dynamic light scattering (DLS), and BET, to determine the physical properties. The magnesium oxide was placed in simulated body fluids (SBFs) with different pH values (5.0 and 7.4) to simulate dissolution rates of the magnesium oxide in tumor and normal human environments.

Results show that the mesoporous nano-magnesium oxide prepared in this example has a particle size of 50 nm to 200 nm, a large number of pores with an average diameter of 14.1 nm distributed on a surface, a total pore volume of 0.431 cm³/g, and a surface potential of 50 mV in absolute ethanol, indicating excellent drug loading performance. Moreover, results of the dissolution rate experiment show that the magnesium oxide has a very low dissolution rate in SBF with a pH of 7.4, but has a high dissolution rate in SBF with a pH of 5.0, indicating that the carrier can be quickly decomposed in a simulated mildly-acidic environment surrounding a tumor while remaining stable in a simulated normal mildly-alkaline environment in the human body.

What is claimed is:

1. A mesoporous nano-magnesium oxide for drug loading, wherein a particle of the mesoporous nano-magnesium oxide for the drug loading has a particle size of 50 nm to 150 nm and comprises abundant mesoporous structures on a surface of the particle of the mesoporous nano-magnesium oxide with a pore size of 2 nm to 20 nm.

2. The mesoporous nano-magnesium oxide for the drug loading according to claim 1, wherein the surface of the particle of the mesoporous nano-magnesium oxide for the drug loading has a positive potential in absolute ethanol, with a Zeta potential distribution of 10 mV to 100 mV.

3. A preparation method of the mesoporous nano-magnesium oxide for the drug loading according to claim 1, comprising the following steps:
(1) a preparation of a magnesium oxalate precursor:
with magnesium chloride hexahydrate as a magnesium source, oxalic acid dihydrate as a precipitating agent, a mixture of deionized water and n-butanol as a solvent, cetyltrimethylammonium bromide (CTAB) as a dispersing agent, and acetic acid added to increase a hydrogen ion concentration, conducting a co-precipitation reaction to synthesize the magnesium oxalate precursor, wherein the magnesium oxalate precursor is a mixture of magnesium oxalate and the CTAB; and
(2) a preparation of a nano-magnesium oxide microparticle
adding the magnesium oxalate precursor to a quartz crucible, and placing the quartz crucible in a muffle furnace; calcining at 200℃ for 1 h to make the CTAB in the mixture completely decomposed to produce pure magnesium oxalate; and heating to 530°C, and calcining for 1 h to 6 h to make the pure magnesium oxalate fully decomposed to produce the nano-magnesium oxide microparticle.

4. The preparation method of the mesoporous nano-magnesium oxide for the drug loading according to claim 3, wherein a concentration ratio of the magnesium chloride hexahydrate to the oxalic acid dihydrate is (1-3):(1-3); and the mixture of the deionized water and the n-butanol in a volume ratio of (1-3):(1-3) is added.

5. The preparation method of the mesoporous nano-magnesium oxide for the drug loading according to claim 3, wherein during the co-precipitation reaction, 0.25 wt% to 1.25 wt% of the CTAB and the acetic acid with a concentration of 36% to 50% are added to improve a dispersibility of the magnesium oxalate.

6. The preparation method of the mesoporous nano-magnesium oxide for the drug loading according to claim 3, wherein a process for preparing the magnesium oxalate precursor is as follows: placing a beaker with a predetermined amount of the solvent in a water bath; when a temperature of the solvent reaches a predetermined reaction temperature, adding a specified concentration ratio of the magnesium chloride hexahydrate and a predetermined amount of the CTAB, thoroughly stirring, and adding specified amounts of the oxalic acid dihydrate and the acetic acid; using an alcohol and the deionized water as detergents; conducting decantation multiple times, and retaining a lower precipitate; and oven-drying to produce the magnesium oxalate precursor.

7. The preparation method of the mesoporous nano-magnesium oxide for the drug loading according to claim 6, wherein the predetermined reaction temperature is 40°C to 80°C, and a reaction time is 20 min to 60 min; the stirring is continuously conducted for 20 min under heating with a magnetic stirrer at a rotational speed of 100 r/min to 1,000 r/min; and after the decantation is conducted multiple times, the oven-drying is conducted at 90°C to 100°C for 1 h.

8. The preparation method of the mesoporous nano-magnesium oxide for the drug loading according to claim 3, wherein with a decomposition of the pure magnesium oxalate, a gas is released from a surface of the nano-magnesium oxide microparticle to form a large number of mesopores and micropores, with an average pore size of 14 nm and a pore size distribution of 2 nm to 20 nm.

9. The preparation method of the mesoporous nano-magnesium oxide for the drug loading according to claim 3, wherein the surface of the particle of the mesoporous nano-magnesium oxide for the drug loading has a positive potential in absolute ethanol, with a Zeta potential distribution of 10 mV to 100 mV.

10. The preparation method of the mesoporous nano-magnesium oxide for the drug loading according to claim 9, wherein a concentration ratio of the magnesium chloride hexahydrate to the oxalic acid dihydrate is (1-3.):(1-3.); and the mixture of the deionized water and the n-butanol in a volume ratio of (1-3.):(1-3.) is added.

11. The preparation method of the mesoporous nano-magnesium oxide for the drug loading according to claim 9, wherein during the co-precipitation reaction, 0.25 wt% to 1.25 wt% of the CTAB and the acetic acid with a concentration of 36% to 50% are added to improve a dispersibility of the magnesium oxalate.

12. The preparation method of the mesoporous nano-magnesium oxide for the drug loading according to claim 9, wherein a process for preparing the magnesium oxalate precursor is as follows: placing a beaker with a predetermined amount of the solvent in a water bath; when a temperature of the solvent reaches a predetermined reaction temperature, adding a predetermined molar mass of the magnesium chloride hexahydrate and a predetermined amount of the CTAB, thoroughly stirring, and adding predetermined amounts of the oxalic acid dihydrate and the acetic acid; using an alcohol and the deionized water as detergents; conducting decantation multiple times, and retaining a lower precipitate; and oven-drying to produce the magnesium oxalate precursor.

13. The preparation method of the mesoporous nano-magnesium oxide for the drug loading according to claim 12, wherein the predetermined reaction temperature is 40°C to 80°C, and a reaction time is 20 min to 60 min; the stirring is continuously conducted for 20 min under heating with a magnetic stirrer at a rotational speed of 100 r/min to 1,000 r/min; and after the decantation is conducted multiple times, the oven-drying is conducted at 90°C to 100°C for 1 h.

14. The preparation method of the mesoporous nano-magnesium oxide for the drug loading according to claim 9, wherein with a decomposition of the pure magnesium oxalate, a gas is released from a surface of the nano-magnesium oxide microparticle to form a large number of mesopores and micropores, with an average pore size of 14 nm and a pore size distribution of 2 nm to 20 nm.
